(19) United States
(12) Patent Application Publication
(10) Pub. No.: US 2010/0024828 A1
(43) Pub. Date: Feb. 4, 2010

(54) LATEX ARTICLES CONTAINING NANO-SILVER AND THE PREPARATION THEREOF

(76) Inventors: Rulin Chen, Guangzhou (CN); Victor WJ Chan, Richmond (CA)

Correspondence Address: Victor WJ Chan, 8020 Saunders Road, Richmond, BC, V7A 2A4, omitted

(21) Appl. No.: 11/916,288

(22) PCT Filed: Nov. 21, 2005
(86) PCT No.: PCT/CN05/01964

§ 371 Date: Jun. 23, 2008

(30) Foreign Application Priority Data
Jun. 3, 2005 (CN) 200510035122.5

(51) Int. Cl. A61F 6/04; B32B 5/02; C08K 3/08; C10M 125/04; A41D 19/015

(52) U.S. Cl. 128/844; 428/338; 524/440; 508/103; 2/167

(57) ABSTRACT

New latex articles containing nano-silver and the preparation thereof are disclosed. The latex articles, which contain nano-silver, are manufactured by molding the latex containing nano-silver or coating the molded latex articles with the lubricant containing nano-silver. The inventive processes are easy and the molded articles can not only be antibacterial, but also be bacteriostasis and bactericidal. So the safety in using it is greatly improved and the cross infection of bacterias is prevented essentially. The said articles are condoms and gloves etc which meets the strict requirement of sanitary.

### FIELD OF TECHNOLOGY TO WHICH THE INVENTION BELONGS

This invention involves a type of latex product and the preparation method thereof.

### PRIOR ART BEFORE THE INVENTION

Ancient or modern, in China or abroad, when human recognition of the natural world was still in the days in which the heaven was considered as round and the earth as flat and square, when human beings were still in the days in which they lacked scientific knowledge of silver but only had visual and perceptual knowledge that resulted from frequent access to and use of various silver coins and silver products in daily life, they already began to use silver ware and silver foil for treatment of diseases and injuries or even used them as health protection medicine. In life, silver is often used to keep fresh and preserve water and food, and to treat injuries and cure wounds in war. In pure water production, silver-loaded active carbon is used to kill bacteria and purify water, and has long been applied in water purifier and various types of pure water purification technology, and the bactericidal mechanism thereof has been sufficiently demonstrated.

Silver is off-white powder, is a relatively stable substance, is basically insoluble in water, in hydrochloric acid and in alkali; and its own bactericidal power is not strong. The known free silver particles or silver groups are the active factors that kill pathogenic bacteria. And silver ions are the charged particles that cannot individually exist in the objective world, and in order to use them, silver ions must be released from a certain preparation before they can be effective. With the progress and development of science, the cycle for updating science and technology has become shorter. Since the rise and rapid development of nano technology in the 1990s, the development of silver in the field of bacterial infection inhibition has entered a new stage. In a period of time of a little more than ten years, the preparation of various types of silver nano particles and their application in the field of bacteria inhibition and infection resistance have witnessed unprecedented development. There are more and more clinical studies and reports on the bactericidal and antitoxic effects of nano silver particles, and the application thereof is also on daily increase. The mechanism for nano silver particles to kill bacteria is that silver particles are able to quickly bind with the sulfhydryl-SH of pheron in the bacteria, and some enzymes dependent on it are thus deactivated and cannot survive, so as to serve, the purpose of killing bacteria and to simultaneously play the role of treatment and prevention against bacteria and fungi.

And for the existing latex products, such as condoms and gloves, cannot inhibit and kill bacteria, and can only play the role of isolating bacteria, and once broken, the effect of protection will be lost, which makes it easy for the user to get infected. Therefore, it has always been a task in the industry for studies on how to manufacture a type of latex product that can inhibit and kill bacteria.

### PURPOSE OF INVENTION

The purpose of this invention is to provide a new type of nano silver latex product and the preparation method thereof, which is simple in preparation, can inhibit and kill bacteria, and greatly enhances the safety in use.

### TECHNICAL SOLUTION USED IN THE INVENTION

The purpose of this invention is implemented in this way: this is a new type of nano silver latex product, and has the following features: the latex product as mentioned contains nano silver particles.

The latex product as mentioned is machine shaped with latex in which nano silver is added.

The latex product as mentioned is made by coating the surface of shaped latex with lubricant in which nano silver is added.

This is the preparation method for a new type of nano silver latex product, and it has the following features: 0.1-1 portion of nano silver particles is weighed and taken on the basis of weight ratio, and is added into 100 portions of latex under agitation, dispersant is added at the same time, and is agitated until it is evenly dispersed to obtain nano silver latex, which is finally machine shaped.

This is the preparation method for a new type of nano silver latex product, and it has the following features: 0.1-1 portion of nano silver particles is weighed and taken on the basis of weight ratio, and is added into 100 portions of lubricant under agitation, dispersant is added at the same time, and is agitated until it is evenly dispersed to obtain nano silver lubricant, and the prepared nano silver lubricant is finally coated evenly on the surface of the shaped latex product.

The invention is simple in preparation technology, and in addition to blocking bacteria, the manufactured product is also bacteriostatic and bactericidal, which has greatly enhanced safety and fundamentally put an end to the possibility for bacteria cross infection, and can thus be widely applied in products such as condoms and gloves that have strict requirements on hygiene.

### DESCRIPTION OF ATTACHED DRAWINGS

FIG. 1 is the diagram for the dispersed state of nano silver particles in latex in the Case of Implementation 1 for the invention;

FIG. 2 is the diagram for the dispersed state of nano silver particles in the silicone oil coated on latex product in the Case of Implementation 2 for the invention.

### CASE OF IMPLEMENTATION

This invention is a new type of nano silver latex product, the latex product contains nano silver particles, and is machine shaped with latex in which nano silver is added, or is made by coating the surface of shaped latex with lubricant in which nano silver is added.

Specifically, it refers to the even dispersion of 1-300 nm nano silver particles in latex (natural latex is preferred) to form nano silver latex, and the nano silver latex is then used to manufacture condoms, gloves and other latex products so as to let nano silver play its bactericidal role. Or 1-300 nm nano silver particles are dispersed in lubricant to form nano silver lubricant, and the nano silver silicone oil or the nano silver lubricant is evenly coated on the surface of condoms, gloves and other latex products, so as to let nano silver play its bactericidal role and serve the purpose of lubrication and killing bacteria. Lubricant can be either water-based or oil-based, silicone oil is preferred, and amino-modified silicone oil is most preferred.

The first of the preparation methods for this invention is that the product is machine shaped with latex in which nano silver is added, the new type of nano silver latex includes natural latex and nano silver particles, and it has the following weight ratio: 100 portions of latex, and 0.1-1 portion of nano silver particles.

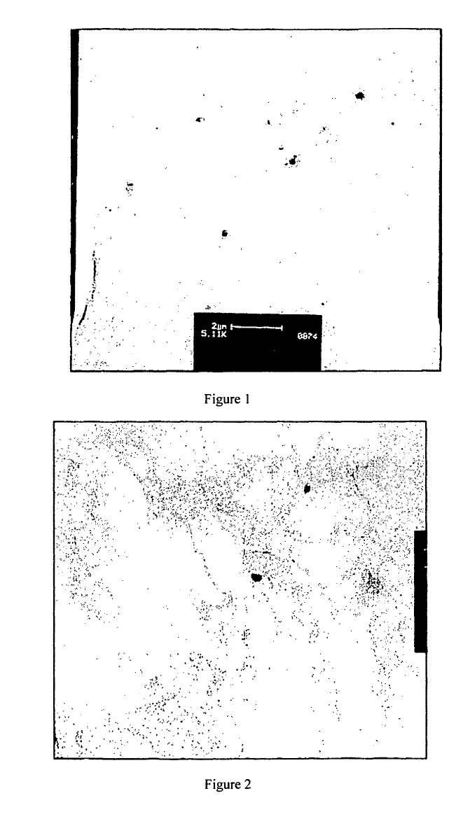

Case of Implementation 1: 0.5 portion of nano silver particles is weighed and taken on the basis of weight ratio, and is added into 100 portions of latex under agitation, an appropriate amount of dispersant is added at the same time, and is agitated until nano silver is completely dispersed evenly in the natural latex, the nano silver natural latex is made into condoms, gloves and other latex products. The latex product is tested through the transmission electron microscope with result as presented in FIG. 1 below, and from FIG. 1, it can be seen that nano silver particles are evenly dispersed in latex.

The second of the preparation methods for this invention is to coat the surface of shaped latex with lubricant in which nano silver is added, including silicone oil (or other water-based or oil-based lubricant) and nano silver particles, which are in the following weight ratio: 100 portions of silicone oil (or other water-based or oil-based lubricant) and 0.1-1 portion of nano silver.

Case of Implementation 2: 0.5 portion of nano silver particles is weighed and taken on the basis of weight ratio, and is added into 100 portions of silicone oil under agitation, an appropriated amount of dispersant is added at the same time, and is agitated until nano silver is completely dispersed in the silicone oil to obtain nano silver silicone oil, and the prepared nano silver silicone oil is evenly mixed and coated on shaped condoms, gloves and other latex products to obtain corresponding nano silver silicone oil condoms, gloves and other latex products. The test result through use of the transmission electron microscope is presented in FIG. 2 below, and from FIG. 2, it can be seen that nano silver particles are evenly dispersed in the silicone oil coated on the latex product.

1. This is a new type of nano silver latex product, and has the following features: the latex product as mentioned contains nano silver particles.

2. With regard to the new type of nano silver latex product as mentioned in claim 1, it has the following features: the latex product as mentioned is machine shaped with latex in which nano silver particles are added.

3. With regard to the new type of nano silver latex product as mentioned in claim 1, it has the following features: the latex product as mentioned is made by coating the surface of shaped latex with lubricant in which nano silver particles are added.

4. (canceled)

5. With regard to the new type of nano silver latex product as mentioned in claim 1, it has the following features: the grain size of the nano silver particle as mentioned is 1-300 nm.

6. With regard to the new type of nano silver latex product as mentioned in claim 3, it has the following features: the lubricant as mentioned silicone oil.

7. With regard to the new type of nano silver latex product as mentioned in claim 6, it has the following features: the silicone oil as mentioned is amino-modified silicone oil.

8. With regard to the new type of nano silver latex product as mentioned in claim 1, it has the following features: the latex products as mentioned are condoms and gloves.

9. This is the preparation method for a new type of nano silver latex product, and it has the following features: 0.1-1 portion of nano silver particles is weighed and taken on the basis of weight ratio, and is added into 100 portions of latex under agitation, dispersant is added at the same time, and is agitated until it is evenly dispersed to obtain nano silver latex, which is finally machine shaped.

10. This is the preparation method for a new type of nano silver latex product, and it has the following features: 0.1-1 portion of nano silver particles is weighed and taken on the basis of weight ratio, and is added into 100 portions of lubricant under agitation, dispersant is added at the same time, and is agitated until it is evenly dispersed to obtain nano silver lubricant, and the prepared nano silver lubricant is finally coated evenly on the surface of the shaped latex product.

11. With regard to the new type of nano silver latex product as mentioned in claim 2, it has the following features: the latex as mentioned is natural latex.

12. With regard to the new type of nano silver latex product as mentioned in claim 3, it has the following features: the latex as mentioned is natural latex.
